(19) United States
(12) Patent Application Publication
(10) Pub. No.: US 2022/0244260 A1
(43) Pub. Date: Aug. 4, 2022

(54) BREAST CANCER DETECTION METHODS

(71) Applicant: Cambridge Enterprise Limited, Cambridge (GB)

(72) Inventors: Walid T. KHALED, Cambridge (GB); Sara PENSA, Cambridge (GB); Karsten BACH, Cambridge (GB)

(73) Assignee: Cambridge Enterprise Limited, Cambridge (GB)

(21) Appl. No.: 17/616,246

(22) PCT Filed: Jun. 4, 2020
(86) PCT No.: PCT/GB2020/051352

§ 371 Date: Dec. 3, 2021

(30) Foreign Application Priority Data
Jun. 5, 2019 (GB) 1907999.5

(51) Int. Cl. G01N 33/574; C12Q 1/6886; C12Q 1/6881; G01N 33/50

(52) U.S. Cl. CPC G01N 33/57415; C12Q 1/6886; C12Q 1/6881; C12Q 2600/158; C12Q 2600/118; G01N 2800/50; C12Q 2600/156; G01N 33/5091

(57) ABSTRACT

The present invention relates to a method of assessing the likelihood of a subject having breast cancer or a precancerous condition associated with breast cancer. The present invention also relates to a method of determining whether treatment of breast cancer in a subject is required, and to a chemotherapeutic agent for use in the treatment of a subject in need of said treatment.

### INTRODUCTION

The present invention relates to a method of assessing the likelihood of a subject having breast cancer or a precancerous condition associated with breast cancer. The present invention also relates to a method of determining whether treatment of breast cancer in a subject is required, and to a chemotherapeutic agent for use in the treatment of a subject in need of said treatment.

### BACKGROUND OF THE INVENTION

Breast cancer is the most common malignant tumour diagnosed in women. In the UK, around 55,000 women are diagnosed with breast cancer every year, with there being approximately 11,000 reported deaths each year in the UK from breast cancer.

Breast cancer is a heterogeneous disease made up of several subtypes. Classification of breast cancer typically involves the use of immunohistological methods to determine the expression level of several key hormone receptors associated with the onset of breast cancer. Of the hormone receptors analysed, the three most notable receptors are the progesterone receptor (PgR), estrogen receptor (ER) and human epidermal growth factor receptor 2 (HER2). Breast cancer patients who express PgR and ER, and also have an overexpressed level of HER2, typically have a good clinical outcome, due to the fact that they are more likely to respond well to hormone therapy, such as, for example, the administration of chemotherapeutic agents that specifically target one or more of these key hormone receptors (e.g. tamoxifen, Arimidex™ (anastrozole), Aromasin™ (exemestane), Femara™ (letrozole), Faslodex™ (fulvestrant), Herceptin™ (trastuzumab) and Tykerb™ (lapatinib)).

One of the most invasive subtypes of breast cancer, accounting for between 15% and 20% of all breast cancers, is triple negative breast cancer (TNBC). TNBC is a type of breast cancer classified by its lack of protein expression of ER, PgR and the absence of HER2 protein over-expression. With PgR, ER and HER2 being absent, TNBC tumours do not respond as well to standard hormone therapy treatment regimens, and therefore TNBC is typically associated with poor prognosis, low survival rates and a high tumour reoccurrence. However, if diagnosed early, TNBC can be successfully treated by surgery, radiotherapy and some chemotherapeutic agents, and further managed or prevented using elective mastectomy and/or chemoprevention.

The prognosis for breast cancer patients, and in particular TNBC patients, is greatly improved if the breast cancer is detected early. Thus, there remains a need for a simple and cost-effective method for detecting breast cancer, particularly for detecting the early onset of breast cancer in women at risk of TNBC.

### SUMMARY OF THE INVENTION

According to a first aspect of the present invention, there is provided a method of assessing the likelihood of a subject having breast cancer or a precancerous condition associated with breast cancer, said method comprising analysing the subject's breast to determine whether aberrantly differentiated luminal progenitor cells are present in the breast, wherein the presence of aberrantly differentiated cells in the breast is indicative of the subject having a high likelihood of having breast cancer or a precancerous condition associated with breast cancer.

Suitably, the aberrantly differentiated luminal progenitor cells are cells that aberrantly differentiate to form alveolar structures (e.g. milk forming structures in the subject's breast).

According to a second aspect of the present invention, there is provided a method of determining whether a subject has breast cancer or a precancerous condition associated with breast cancer, said method comprising analysing the subject's breast to determine whether aberrantly differentiated luminal progenitor cells and/or alveoli structures are present in the breast, wherein the presence of aberrantly differentiated cells and/or alveoli structures in the breast is indicative of the subject having breast cancer or a precancerous condition associated with breast cancer.

According to a third aspect of the present invention, there is provided a method of treating breast cancer in a subject, said method comprising the steps of:
a) assessing the likelihood of the subject having breast cancer or a precancerous condition associated with breast cancer by a method as described herein; and
b) administering a standard breast cancer treatment regimen to the subject if the likelihood of the subject having breast cancer or a precancerous condition associated with breast cancer is assessed as being high.

According to a fourth aspect of the present invention, there is provided a method of monitoring the onset of breast cancer or a precancerous condition associated with breast cancer, or monitoring the recurrence and/or growth of breast cancer in a subject, said method involving assessing the likelihood of the subject having breast cancer or a precancerous condition associated with breast cancer, using a method as defined herein at two or more time points separated by a predetermined time interval.

According to a fifth aspect of the present invention, there is provided a chemotherapeutic agent for use in the treatment of a subject in need of said treatment, the treatment comprising the steps of:
a) assessing the likelihood of the subject having breast cancer or a precancerous condition associated with breast cancer by a method as described herein;
b) administering a chemotherapeutic agent to the subject if the likelihood of having breast cancer or a precancerous condition associated with breast cancer is assessed as being high.

According to a sixth aspect of the present invention, there is provided a method of assessing the likelihood of a subject having breast cancer or a precancerous condition associated with breast cancer, said method comprising analysing a sample of material obtained from the subject's breast to determine whether beta-casein (CSN2) is present, wherein the presence of beta-casein (CSN2) in the sample of material obtained from the subject's breast is indicative of the subject having a high likelihood of having breast cancer or a precancerous condition associated with breast cancer.

When an assessment of the likelihood of a subject having breast cancer is carried out with a high level of certainty, the method of the invention is a method of determining whether the subject has breast cancer.

In preferred embodiments, therefore, a method of assessing the likelihood of a subject having breast cancer is a method of determining whether the subject has breast cancer.

### DESCRIPTION OF THE DRAWINGS

FIG. 1 shows: A) the percentage of cells within the luminal (left) or basal (right) compartment of the mammary glands collected from Bcl11a labelled mice (e.g. mice administered tamoxifen (TAM)) at 8 weeks post labelling (8 w) and 14.5 days gestation (14.5 dG), as determined by FACS, wherein the data is represented as mean±SD and an unpaired t-test was performed (* indicates P<0.05); and B) confocal 3D images of tdTomato^{pos} cells of CUBIC-cleared whole glands immunostained with EpCAM or K5 as indicated and shown as overview (top 2 panels) or representative Z-slices (bottom 2 panels) of mammary epithelial structures; scale bars, 100 μm.

FIG. 2 shows: A) a schematic representation of the MPA/DMBA protocol used to induce TNBC tumours in mice after induction of Bcl11a labelling; and B) confocal 3D images of tdTomato^{pos} cells of CUBIC-cleared whole glands immunostained with EpCAM or smooth muscle actin (SMA) as indicated and shown as representative Z-slices of mammary epithelial structures; scale bars, 100 μm.

FIG. 3 shows a Kaplan-Meier survival plot depicting tumour free survival of Brca1^{f/f}; p53^{+/-}; Blg-Cre (Bdl11a^{+/+ or f/+}) (n=14) and Bcl11a^{f/f}; Brca1^{f/f}; p53+^{+/-}; Blg-Cre (n=6). Log rank (Mantel-cox) test performed, p=0.00017.

FIG. 4 shows t-distributed stochastic neighbor embedding (t-SNE) representations of mammary epithelial cells from Brca1^{f/f}; p53^{+/-}; Blg-Cre (left panel) and Bcl11a^{f/f}; Brca1^{f/f}; p53^{+/-}; Blg-Cre (right panel) mice that were isolated by FACS and subjected to droplet-based scRNAseq. Cells are coloured based on inferred cell types.

FIG. 5 shows representative mammary wholemounts from virgin control, BCL11A^{ovx}; MMTV-Cre, BCL11A^{ovx}; Blg-Cre, Brca1^{f/f}; p53^{+/-}; Blg-Cre and Bcl11a^{f/f}; Brca1^{f/f}; p53^{+/-}; Blg-Cre mice from various ages.

FIG. 6 shows: A) a graph depicting the average protein expression of CSN2 in BRCA1 mutation carriers and non-BRCA1 carriers (normal) measured by IHC; and B) shows a graph depicting the average RNA expression of CSN2 in BRCA1 mutation carriers and non-BRCA1 carriers (normal) measured by qPCR.

FIG. 7 shows: A) Schematic: 7 weeks after induction of Bcl11a labelling by a single intraperitoneal injection of a low dose of tamoxifenduring puberty, mice were subjected to the MPA/DMBA protocol to induce TNBC tumours and samples were collected 11 weeks later; B) tilescan of a mammary gland section immunostained with RFP (to detect tdTomatopos cells) and DAPI, scale bars, 100 μm; C) confocal 3D imaging of tdTomatopos cells of CUBIC-cleared whole glands immunostained with EpCAM shown as frame shot of a 3D reconstruction of 2 representative mammary epithelial structures; scale bars, 100 μm; D) mammary gland sections immunostained with RFP (to detect tdTomatopos cells), Csn2 or DAPI, scale bars, 100 μm; and E) schematic depicting RNA extraction from luminal progenitor cells that were FACS isolated at various timepoints following MPA/DM BA treatment, with the graph showing Csn2 expression in the various samples.

FIGS. 8 and 9 show extracts from the LC-MS analysis of mammary gland tissue from Brca1/p53 mice. FIG. 8 shows the mass spectrum of a peptide matching CSN2. FIG. 9 shows the mass spectrum of a peptide matching WAP.

FIGS. 10 and 11 show extracts from the LC-MS analysis of blood from lactating WT mice. FIG. 10 shows the mass spectrum of a peptide matching CSN2. FIG. 11 shows the mass spectrum of a peptide matching WAP.

### DETAILED DESCRIPTION OF THE INVENTION

### Methods of the Invention

### Likelihood of a Subject Having Breast Cancer

Following extensive investigation, the inventors have surprisingly discovered that luminal progenitor cells aberrantly differentiate in the breasts of both animal and human subjects diagnosed with breast cancer (e.g. TNBC), often well in advance of any visible signs of tumour development. Luminal progenitor cells are the cells that are present in a women's breast(s) and typically only differentiate to form alveoli structures (e.g. milk producing cells) during pregnancy. Thus, it was to the inventors surprise that such cells were found to be aberrantly differentiated in the early stages of breast cancer (in non-pregnant subjects).

In view of this discovery, the present invention provides a novel method for determining the likelihood of a patient having breast cancer, which uses the detection of aberrantly differentiated luminal progenitor cells (and/or the detection of alveoli structures) in a subject's breast. Furthermore, as the differentiation of the luminal progenitor cells, and the subsequent formation of alveoli structures were typically observed in advance of any visible signs of tumour development, the present invention also advantageously provides a method for the detection of early forms of breast cancer.

Thus, in one aspect of the present invention, there is provided a method of assessing the likelihood of a subject having breast cancer or a precancerous condition associated with breast cancer, said method comprising analysing the subject's breast to determine whether aberrantly differentiated luminal progenitor cells are present, wherein the presence of aberrantly differentiated cells is indicative of the subject having a high likelihood of having breast cancer or a precancerous condition associated with breast cancer. The absence of aberrantly differentiated cells is indicative of the subject having a low likelihood of having breast cancer or a precancerous condition associated with breast cancer.

In certain embodiments, the method comprises analysing the subject's breast to determine whether aberrantly differentiated luminal progenitor cells and/or alveoli structures are present in the breast, wherein the presence of aberrantly differentiated cells and/or presence of alveoli structures is indicative of the subject having a high likelihood of having breast cancer or a precancerous condition associated with breast cancer.

Suitably, the luminal progenitor cells are alveolar luminal progenitor cells, for example, alveolar luminal progenitor cells aberrantly differentiated to form alveolar structures.

In certain embodiments, the method of the present invention involves assessing the likelihood of a subject having breast cancer. Most suitably, the method of the present invention involves assessing the likelihood of a subject having triple negative breast cancer (TNBC).

In some embodiments, the method of the present invention involves assessing the likelihood of a subject having a precancerous condition associated with breast cancer. Precancerous conditions being conditions in which the morphology of certain cells is disordered, thereby increasing the risk of the breast cancer in the subject.

### Breast Analysis

Analysis of the subject's breast to determine whether aberrantly differentiated luminal progenitor cells and/or alveoli structures are present may involve any suitable technique known in the art. In certain embodiments, the analysis of the subject's breast comprises scanning and/or imaging the breast to determine whether aberrantly differentiated cells and/or alveoli structures are present. In other embodiments, the method of the present invention may comprise the step of obtaining a sample of material from the breast of the subject, and analysing the material to determine whether one or more species associated with aberrantly differentiated cells are present.

Thus, in certain embodiments, the analysis of the subject's breast comprises one or more techniques selected from:
a) scanning and/or imaging the breast to determine whether aberrantly differentiated luminal progenitor cells are present;
b) and/or obtaining a sample of material from the breast of the subject, and analysing the material to determine whether a species associated with aberrantly differentiated luminal progenitor cells is present.

The scanning and/or imaging the breast may be performed using any suitable technique known in the art. Suitably, the scanning and/or imaging the breast is conducted using one or more techniques selected from magnetic resonance imaging (MRI), mammography, computerised tomography (CT) scanning, and/or contrast imaging.

Species that are associated with the aberrantly differentiated luminal progenitor cells may include, for example, proteins, DNA molecules and/or RNA molecules. Suitably, the species that is associated with the aberrantly differentiated luminal progenitor cells is selected from the group consisting of milk proteins and/or DNA or RNA molecules that codes for milk proteins.

In certain embodiments, the species that is associated with the aberrantly differentiated luminal progenitor cells is a milk protein. Suitably, the milk protein is selected from casein (e.g. beta-casein), alpha-lactalbumin, lactoferrin, immunoglobulin immunoglobulin A, lysozyme, whey acidic protein (WAP) and serum albumin. Most suitably, the milk protein is casein (e.g. beta-casein) or whey acidic protein. Even more suitably, the milk protein is beta-casein or WAP. Most suitably, the milk protein is beta-casein.

In other embodiments, the species associated with the aberrantly differentiated luminal progenitor cells is a DNA or RNA molecule that codes for a milk protein. Suitably, the DNA or RNA molecule that codes for a milk protein is selected from alpha S1-casein (CSN1S1), beta-casein (CSN2), kappa-casein (CSN3), whey acidic protein and beta-lactoglobulin (LGB). More suitably, the DNA or RNA molecule that codes for a milk protein is whey acidic protein (WAP) or beta-casein (CSN2). Most suitably, the DNA or RNA molecule that codes for a milk protein is beta-casein (CSN2).

The analysis of the material obtained from a subject's breast to determine whether a species associated with the aberrantly differentiated luminal progenitor cells is present may be performed using any suitable technique known in the art.

In embodiments where the analysis of the material obtained from a subject's breast involves determining whether a protein is present, the analysis may be performed using one or more techniques selected from enzyme-linked immunosorbent assay (ELISA), Western Blot analysis and mass spectrometry.

In embodiments where the analysis of the material obtained from a subject's breast involves determining whether a DNA or RNA molecule is present, the analysis may be performed using one or more techniques selected from quantitative polymerase chain reaction (qPCR), RNA sequencing (RNAseq) and DNA sequencing (DNAseq).

The sample of material from the breast of the subject may be a sample obtained from the breast via biopsy or surgery, or from a sample of material expressed from the breast (e.g. a fluid expressed from the nipple of the breast), or from a sample of blood taken from the subject.

Suitably, the sample of material from the breast of the subject is a fluid which has been expressed from the breast of the subject. Most suitably, the sample of material from the breast of the subject is a fluid that has been expressed via the nipple of the subject's breast. The sample of (expressed) material may be obtained using any technique known in the art, such as, for example, extraction via a breast pump and/or extraction via a needle.

Suitably, the sample of material from the breast of the subject is a sample obtained from a sample of blood taken from the subject. It will be understood that the sample of material from the breast of a subject may be material that enters the blood of the subject from the breast (e.g. from the mammary gland). As such, the sample of blood referred to above, from which the sample of material from the breast is obtained, may be taken from anywhere on the body of the subject, not necessarily the breast of the subject.

In certain embodiments, the method of the present invention comprises the step of analysing material obtained from the breast of the subject to determine whether one or more further species commonly associated with breast cancer and/or aberrantly differentiated luminal progenitor cells are present, or, as appropriate, are present in a higher level than in a normal (control) subject. The one or more further species may be, for example, oncogenes commonly associated with breast cancer. Suitably, the one or more further species is a DNA or RNA molecule from a gene selected from BCL11A, ELF5, SOX10, FOXC1 Ki-67, HOXD13 and PCDHGB7, and/or a protein expressed from said gene. Most suitably, the one or more further species is selected from a DNA or RNA molecule from a gene selected from BCL11A, ELF5, SOX10, FOXC1 and Ki-67, and/or a protein expressed from said gene. Even more suitably, the one or more further species is selected from a DNA or RNA molecule from a gene selected from BCL11A, SOX10, and Ki-67, and/or a protein expressed from said gene. Most suitably, the one or more further species is a DNA or RNA molecule from BCL11A, and/or the Bcl11a protein.

The inventors discovered there to be a strong correlation between the breast cancer oncogene, BCL11A, and the aberrant differentiation of luminal progenitor cells. For example, it was discovered that deletion of Bcl11a resulted in the protection against aberrant differentiation of the luminal progenitor cells in a Brca1/p53 TNBC mouse model (as described in the example below), and, in some cases, protection against the development of breast tumours.

The inventors further discovered that in the aberrantly differentiated luminal progenitor cells a number of genes were upregulated. The genes the inventors discovered were significantly upregulated in the aberrantly differentiated luminal progenitor cells included Csn2, Csn1s1, B2m, Csn1s2a, H2.Q7, Bst2, Tceal9, Plin2, Muc15, Fth1, H2.K1, Mt1, Hspe1, Tmem176b, Mt2, Wfdc18, Sec61b, Vamp8, Tubb5, Ubd, Ogfrl1, Stmn1, Rbp1, H2.Q6, Dbi, Elf5, Psmb8, Chchd2, Serf2, Prdx2, Calr, Atp5h, Mgst1, Fam3c, H2afz, Pdk4, Ndufb9, Ubald2, Cox8a, Marcksl1, Cox6a1, H2.D1, Sod2, H3f3a, Tmem176a, Cox7b, Atp5o, Cox6b1, Psmb3, Pdia6, C3, Cox4i1, Ctsz, Gapdh, Mif, Atp5j2, Psma7, Clta, Sat1, Plscr1, Hspa8, Fkbp11, Scd1, Acot1, Mrps21, Cenpx, Cox5b, Cox6c, Pebp1, Ndufc2, Atp5c1, Ifitm3, Hspd1, Mbd1, Dnajal, Pomp, Angptl4, Tecr, Ssr2, Cib1, Clqtnf1, Cldn3, Cd74, Cyba, P4hb, Map1lc3b, 1700025G04Rik, Calm2, Psmb1, Hsp90ab1, Eef1g, Ndufb6, Atf3, Jpt1, Ndufa4, H2.Q4, Hsp90b1, Pdia3, Cuta, Fkbp2, Ssr4, H2afv, Ndufb11, Tmem258, Selenof, Cox5a, Sox4, Zfand5, mt.Co2, Prdx1, Atpif1, Uqcr10, Serp1, Nop10, Psmb4, Ndufa7, Ndufs6, Atp5j, Ndufa2, Rspo1, Taldo1, Ndufa11, Hspa5, Eci2, Uqcr11, Ndufb3, Eno1, Ehf, H2.T22, Cox7a2, Cks1b, Hist1h2bc, Npc2, Plscr2, Atp5g3 and Sdf2l1.

Thus, in certain embodiments, the method of the present invention comprises the step of analysing material obtained from the breast of the subject to determine whether one or more of the following genes are present, or upregulated: Csn2, Csn1s1, B2m, Csn1s2a, H2.Q7, Bst2, Tceal9, Plin2, Muc15, Fth1, H2.K1, Mt1, Hspe1, Tmem176b, Mt2, Wfdc18, Sec61b, Vamp8, Tubb5, Ubd, Ogfrl1, Stmn1, Rbp1, H2.Q6, Dbi, Elf5, Psmb8, Chchd2, Serf2, Prdx2, Calr, Atp5h, Mgst1, Fam3c, H2afz, Pdk4, Ndufb9, Ubald2, Cox8a, Marcksl1, Cox6a1, H2.D1, Sod2, H3f3a, Tmem176a, Cox7b, Atp5o, Cox6b1, Psmb3, Pdia6, C3, Cox4i1, Ctsz, Gapdh, Mif, Atp5j2, Psma7, Clta, Sat1, Plscr1, Hspa8, Fkbp11, Scd1, Acot1, Mrps21, Cenpx, Cox5b, Cox6c, Pebp1, Ndufc2, Atp5c1, Ifitm3, Hspd1, Mbd1, Dnajal, Pomp, Angptl4, Tecr, Ssr2, Cib1, C1qtnf1, Cldn3, Cd74, Cyba, P4hb, Map1lc3b, 1700025G04Rik, Calm2, Psmb1, Hsp90ab1, Eef1g, Ndufb6, Atf3, Jpt1, Ndufa4, H2.Q4, Hsp90b1, Pdia3, Cuta, Fkbp2, Ssr4, H2afv, Ndufb11, Tmem258, Selenof, Cox5a, Sox4, Zfand5, mt.Co2, Prdx1, Atpif1, Uqcr10, Serp1, Nop10, Psmb4, Ndufa7, Ndufs6, Atp5j, Ndufa2, Rspo1, Taldo1, Ndufa11, Hspa5, Eci2, Uqcr11, Ndufb3, Eno1, Ehf, H2.T22, Cox7a2, Cks1b, Hist1h2bc, Npc2, Plscr2, Atp5g3 and Sdf2l1.

Suitably, the method of the present invention comprises the step of analysing material obtained from the breast of the subject to determine whether one or more of the following genes are present, or upregulated: Csn2, Csn1s1, B2m, Csn1s2a, H2.Q7, Bst2, Tceal9, Plin2, Muc15, Fth1, H2.K1, Mt1, Hspe1, Tmem176b, Mt2, Wfdc18, Sec61b, Vamp8, Tubb5, Ubd, Ogfrl1, Stmn1, Rbp1, H2.Q6, Dbi, Elf5 and Psmb8.

More suitably, the method of the present invention comprises the step of analysing material obtained from the breast of the subject to determine whether one or more of the following genes are present, or upregulated: Csn2, Csn1s1, B2m, Csn1s2a, H2.Q7, Bst2, Tceal9, Plin2, Muc15, Fth1, H2.K1, Mt1, Hspe1, Tmem176b, Mt2, Wfdc18, Sec61b, Vamp8 and Tubb5.

Even more suitably, the method of the present invention comprises the step of analysing material obtained from the breast of the subject to determine whether one or more of the following genes are present, or upregulated: Csn2, Csn1s1, B2m, Csn1s2a, H2.Q7, Bst2 and Tceal9.

Most suitably, the method of the present invention comprises the step of analysing material obtained from the breast of the subject to determine whether one or more of the following genes are present, or upregulated: Csn2, Csn1s1, B2m and Csn1s2a.

The present invention may also include the step of analysing material obtained from the breast of the subject to determine whether one or more proteins expressed from the above mentioned genes are present (or overexpressed).

In one embodiment, there is provided a method of assessing the likelihood of a subject having breast cancer or a precancerous condition associated with breast cancer, said method comprising analysing a sample of material from the breast of the subject obtained from a sample of blood taken from the subject to determine whether beta-casein (CSN2) is present, wherein the presence of beta-casein (CSN2) in the sample of material obtained from the subject's breast is indicative of the subject having a high likelihood of having breast cancer or a precancerous condition associated with breast cancer.

### Detection Techniques

As outlined above, the present invention provides a novel method for determining the likelihood of a subject having breast cancer or a precancerous condition associated with breast cancer. The present invention therefore may beneficially be used together with existing methods available to medical practitioners for assessing the likelihood of a subject having breast cancer (e.g. TNBC). The present invention therefore advantageously provides an additional technique that medical practitioners can use to help assess the likelihood of a subject having breast cancer or a precancerous condition associated with breast cancer.

The techniques commonly employed by medical practitioners for determining the likelihood of a subject having a breast cancer are well known to the person skilled in the art and include, for example, an assessment of a subject's medical history and/or the medical history of the subject's family, examination of the patient, imaging the whole or part of the breast, testing the subject's blood for breast cancer biomarkers, genetically testing the subject's DNA and/or RNA and assessing biopsies taken from the subject. Such techniques are routinely used to determine the presence of risk factors that are commonly associated with breast cancer (e.g. a family history of breast cancer, genetic defect/mutations commonly associated with breast cancer (e.g. mutations to the BRCA1 gene), and/or atypical growths/tumours in the subject's breast).

In some embodiments, the method of the present invention may include using one or more of the aforementioned techniques, in addition to method described hereinabove, such that the presence of one or more risk factors detected using one or more of the aforementioned techniques, in addition to the presence of aberrantly differentiated cells and/or alveoli structures in the subject's breast guides the indication of whether or not the subject is likely to have breast cancer (e.g. TNBC).

### The Subject

It will be understood that the subject may be a human or animal subject. Preferably, the subject is a human subject, and the breast cancer or precancerous conditions is a human breast cancer or precancerous condition associated with a human breast cancer.

In certain preferred embodiments, the subject is a non-pregnant subject. Thus, suitably, the subject is one who has undertaken a pregnancy test, and, most suitably, is a subject one who has undertaken a pregnancy test and had a negative result.

In some embodiments, the subject is a subject who has been identified as being at risk of developing triple negative breast cancer. Thus, suitably, the subject is one who has one or more risk factors commonly associated with breast cancer (e.g. TNBC). Risk factors may include, for example, a family history of breast cancer, pains or discomforts of the breast, atypical growths on or around the breast, swelling or enlargement of one or both breasts, dense breast tissue, mutation of the BRCA1 and/or BRCA2 gene, and homologous recombination deficient. Suitably, the subject is one who has a mutation of the BRCA1 and/or BRCA2 gene, and most suitably, a mutation of the BRCA1 gene.

### Treatment of Breast Cancer

According to a third aspect of the present invention, there is provided a method of treating breast cancer in a subject, said method comprising the steps of:
a) assessing the likelihood of the subject having breast cancer or a precancerous condition associated with breast cancer by a method as described herein; and
b) administering a standard breast cancer treatment regimen to the subject if the likelihood of the subject having breast cancer or a precancerous condition associated with breast cancer is assessed is high.

The one or more standard tumour treatment regimens can be any suitable treatment regimen commonly employed to treat a breast cancer or a precancerous condition associated with breast cancer. Suitably, the one or more standard tumour treatment regimens are selected from surgery, radiotherapy, chemotherapy, immunotherapy and a combination thereof. Most suitably, a standard tumour treatment regimen is selected from surgery, chemotherapy or radiotherapy and a combination thereof.

Surgery may be the surgical intervention by a medical practitioner to remove the whole or part of a tumour (and or breast). Non-limiting examples of suitable surgical intervention include biopsy, incisions, drainage and removal of the breast. Surgery also includes preventative surgical interventions, such as, for example, elective mastectomy.

Radiotherapy may be any form of treatment utilising ionizing radiation. Non-limiting examples of suitable radiotherapy treatments include, for example, external beam radiotherapy (EBRT), stereotactic radiosurgery (STRS) and teletherapy. Suitably, the radiotherapy is external beam radiotherapy (EBRT).

Chemotherapy may be a treatment by administration of one or more anti-tumour (anti-cancer) agents. Non-limiting examples of suitable anti-tumour agents include, for example, tamoxifen, Arimidex™ (anastrozole), Aromasin™ (exemestane), Femara™ (letrozole), Faslodex™ (fulvestrant), Herceptin™ (trastuzumab), Tykerb™ (lapatinib), an anthracycline (e.g. doxorubicin or epirubicin), a taxane (e.g. docotaxel or paclitaxel) and combinations thereof. Suitably, the anti-tumour agents is an anthracycline (e.g. doxorubicin or epirubicin), a taxane (e.g. docotaxel or paclitaxel), or combinations thereof.

Chemotherapy may also include chemoprevention and the administration of agents such as, for example, tamoxifen and raloxifene, which are commonly used to prevent and/or manage the onset of breast cancer (e.g. TNBC).

Immunotherapy may be any form of treatment which exploits the patient's immune system. Non-limiting examples of suitable immunotherapeutic treatment includes, for example, treatments utilising one or more of the following: monoclonal antibodies (MABs); vaccinations; cytokines; and CAR T-cells.

### Active Surveillance

Some breast cancers, and precancerous conditions associated with breast cancer, grow very slowly. Thus, in such circumstances, active surveillance of a subject may be required to monitor a subject's breast over time to check whether breast cancer (or a precancerous condition associated with breast cancer) develops and/or grows.

Active surveillance is particularly useful for subjects who have been identified as being at risk of developing breast cancer (e.g. triple negative breast cancer), such as, for example, subjects who have a mutation of the BRCA1 and/or BRCA2 gene, or a family history of breast cancer, or who subjects who is homologous recombination deficient.

Thus, according to a fourth aspect of the present invention, there is provided a method of monitoring for the onset of breast cancer or a precancerous condition associated with breast cancer, or for monitoring the recurrence and/or growth of breast cancer in a subject, said method involving assessing the likelihood of the subject having breast cancer or a precancerous condition associated with breast cancer by a method as defined herein at two or more time points separated by a predetermined time interval.

It will be appreciated that the number and duration of predetermined time interval may vary depending on the intended application of the method. For example, the pre-determined time interval used may vary depending on the type of breast cancer being detected, the type and sensitivity of instrument being used for the analysis, and/or the subject the analysis is being performed on. Such variations to the pre-determined time interval may be routinely determined by a person skilled in the art based on the intended application of the methodology.

In certain embodiments of the method for monitoring onset of breast cancer or a precancerous condition associated with breast cancer, or for monitoring the recurrence and/or growth of breast cancer in a subject of the present invention, the pre-determined time interval is every 1 to 12 months (e.g. every 1 to 6 months, every 1 to 3 months, every 1 to 2 months or every month).

In other embodiments of the method for monitoring onset of breast cancer or a precancerous condition associated with breast cancer, or for monitoring the recurrence and/or growth of breast cancer in a subject of the present invention, the pre-determined time interval is every 1 to 4 weeks (e.g. every 1 to 3 weeks, every 1 to 2 weeks or every week).

In other embodiments of the method for monitoring onset of breast cancer or a precancerous condition associated with breast cancer, or for monitoring the recurrence and/or growth of breast cancer in a subject of the present invention, the pre-determined time interval is every 1 to 14 day (e.g. every 1 to 7 days, every 1 to 3 days or every day).

### Chemotherapeutic Agents

According to a fifth aspect of the present invention, there is provided a chemotherapeutic agent for use in the treatment of a subject in need of said treatment, the treatment comprising the steps of:
a) assessing the likelihood of the subject having breast cancer or a precancerous condition associated with breast cancer by a method as described herein;
b) administering a chemotherapeutic agent to the subject if the likelihood of the subject having breast cancer or a precancerous condition associated with breast cancer is assessed as being high.

In certain embodiments, the chemotherapeutic agent is selected from tamoxifen, Arimidex™ (anastrozole), Aromasin™ (exemestane), Femara™ (letrozole), Faslodex™ (fulvestrant), Herceptin™ (trastuzumab), Tykerb™ (lapatinib), an anthracycline (e.g. doxorubicin or epirubicin) or a taxane (e.g. docotaxel or paclitaxel). Suitably, the chemotherapeutic agent is an anthracycline (e.g. doxorubicin or epirubicin) or a taxane (e.g. docotaxel or paclitaxel).

Preferred and suitable features relating to the first aspect of the invention, described hereinabove, also represent the preferred and suitable features of all further aspects of the present invention. Thus, features, integers, characteristics, or properties described in conjunction with a particular aspect, embodiment or example of the invention are to be understood to be applicable to all aspects, embodiments or examples described herein unless incompatible therewith. All of the features disclosed in this specification (including any accompanying claims, abstract and drawings), and/or all of the steps of any method so disclosed, may be combined in any combination, except combinations where at least some of such features and/or steps are mutually exclusive.

It will be understood that throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

### EXAMPLES

### Materials and Methods

### Mouse Experiments

All experimental animal work was performed in accordance to the Animals (Scientific Procedures) Act 1986, UK and approved by the Ethics Committee at the Sanger Institute. The Brca1^{f/f}; p53^{+/-}; Blg-Cre (JAX 012620) (J Pathol., 211, 389-398 (2007)) were used to study TNBC tumour development. These mice were crossed to Bcl11a^{f/f} mice to generate Bcl11a^{f/f}; Brca1^{f/f}; p53^{+/-}; Blg-Cre mice. Bcl11a knock-out in the luminal progenitors was achieved by crossing Bcl11a^{f/f} to B/g-Cre mice (Transgenic Res., 7, 387-396 (1998)). The Rosa26-LSL-BCL11A^{ovx} mice were described recently (Nat. Commun., 0, 3327, (2018)). Briefly, the ROSA26 allele was targeted with a construct containing human BCL11A cDNA preceded by a loxP flanked STOP cassette and marked eGFP under the control of an internal ribosomal entry site (IRES) downstream of the inserted cDNA and transgene transcription is controlled by the CAGG promoter. These mice were crossed to the Brca1^{f/f}; p53^{+/-}; Blg-Cre to study the effect of BCL11A overexpression on TNBC tumour development. The Bcl11a^{CreERT2} allele was generated by introducing a IRES-CreERT2-polyA-FRT-PGK-E7-Neo-polyA-FRT cassette into the 3′UTR of Bcl11a locus. Gene targeting was carried out in AB2.2 cells (129 background). Correct targeting was confirmed by long-range PCR and targeted clones were used for blastocyst injections. Chimeric mice were then crossed to wildtype mice (C57/B6) and germline transmission was confirmed. The Bcl11a^{CreERT2} line was then crossed to the Rosa26-LSL-tdTomato mouse line (JAX 007905)²⁵ to generate double transgenic mice. For each experiment, the animals used were heterozygous for Bcl11a^{CreERT2} and homozygous for Rosa26-LSL-tdTomato. Lineage tracing was induced at puberty (5 weeks of age) or in the adult (9 weeks of age) with either 1 single intraperitoneal injection or 3 injections on 3 consecutive days of tamoxifen (1 mg per injection) in corn oil. We confirmed by qRT-PCR that tamoxifen administration leads to true labelling of Bcl11a expressing cells in the mammary gland. Tissues were collected at the indicated time points post injection. For the gestation time point, females were mated with studs 8 weeks after tamoxifen injection and tissues were harvested at gestation day 14.5. For each time point Bcl11a^{CreERT2} wildtype control littermates were injected at the same time as experimental animals and tissues collected to set background levels of tdTomato fluorescence both in FACS and whole tissue IF experiments. The scRNA sequencing experiment was set up to allow for all of the animals to be collected and tissues to be processed at the same time and two individual mice per genotype were used in the study. The Oestrus cycle stage of nulliparous animals was determined by vaginal smears. All mice were housed in individually ventilated cages under a 12:12 h lightdark cycle, with water and food available ad libitum and euthanized by terminal anaesthesia.

Mammary Gland Dissociation into Single-Cell Suspension

Lymph node divested mammary glands (excluding the cervical pair) were dissected from the mice and digested 0/N in DMEM/F12 (Gibco)+10 mM HEPES (Gibco)+1 mg ml⁻¹ collagenase (Roche)+100 U ml⁻¹ hyaluronidase (Sigma) (CH)+gentamicin (Gibco) at 37° C. For scRNA sequencing experiments, the mammary glands were instead mechanically dissociated after collection and the finely minced tissue was transferred to a digestion mix containing double amount of CH for 3 h at 37° C. and vortexed every 30 min. After the lysis of red blood cells in NH₄Cl, cells were briefly digested with warm 0.05% Trypsin-EDTA (Gibco), 5 mg ml⁻¹ dispase (Sigma) and 1 mg ml⁻¹ DNase (Sigma) and filtered through a cell strainer (BD Biosciences).

### Cell Labelling Followed by Flow Cytometry and Sorting

Single-cell suspensions were incubated in HF medium (Hank's balanced salt solution (Gibco)+1% fetal bovine serum, Gibco)+10% normal rat serum (Sigma) for 20 min on ice to pre-block before antibody staining. All antibody incubations were performed for 10 min on ice in HF media. Mouse mammary cells were stained with the following primary antibodies: Cd31-biotin (eBioscience, clone 390, 1 μg ml⁻¹, 1:500); Cd45-biotin (eBioscience, clone 30F11, 1 μg ml⁻¹, 1:500); Ter119-biotin (eBioscience, clone Ter119, 1 μg ml⁻¹, 1:500), EpCAM-APC/Cy7 (Biolegend, clone G8.8, 0.5 μg ml⁻¹, 1:500), Cd49f-BV421 (Biolegend 313623, 2 μg ml⁻¹, 1:100), Cd49b-AF488 (Biolegend, clone HMa2, 1 μg ml⁻¹, 1:500) and Sca1-AF647 (Biolegend, clone D7, 1 μg ml⁻¹, 1:500). Cells were then stained with streptavidin-APC or Streptavidin-PE/Cy7 (BD-Biosciences, 0.4 μg ml⁻¹, 1:500). 7-AAD (Sigma, 10 μg ml⁻¹, 1:100) or Zombie Aqua (Biolegend, 1:100) were used to detect dead cells. Human mammary cells were stained with the following primary antibodies: CD45-APC (Biolegend, clone H130,1:100), CD31-APC (Biolegend, clone WM-59, 1:100), EPCAM-APC/Fire750 (Biolegend, clone 9C4, 1:50), CD49f-PE/Cy7 (Biolegend, clone GoH3, 1 μg ml⁻¹, 1:200). DAPI was used to detect dead cells. Cells were filtered through a cell strainer (Partec) before sorting. Sorting of cells was done using a SH800Z sorter (SONY) after sorting calibration was performed with automatic setup beads (SONY) immediately prior to sorting, or with a FACS Aria Fusion. Single-stained control cells were used to perform compensation manually. Unstained cells and control animals were used to set gates. After doublets, dead cells and contaminating haematopoietic, endothelial and stromal cells were gated out. SH800Z SONY software or FlowJo were used to analyse FACS data. Mouse cells were analysed for tdTomato expression in mammary luminal or basal compartments or luminal cells were sorted for luminal progenitor CFC assays. For scRNAseq, EpCAM-positive cells were sorted in LoBind microcentrifuges tubes (Eppendorf) with HF. After sorting, cells were spun down and resuspended in HF. Samples were manually counted using an improved Neubauer chamber and the cell concentration was normalised by addition of HF. Equal numbers of cells per sample were processed for scRNA library preparation. Samples were processed for library preparation within 9 h from tissue isolation. Human luminal progenitors were sorted for RNA processing.

### Luminal Progenitor CFC Assay

For luminal progenitor colony-forming assays, luminal tdTomato^{pos} or tdTomato^{neg} cells were sorted and plated with irradiated feeders in EpiCult Medium (StemCell) supplemented with 5% fetal bovine serum (StemCell), 10 ng ml⁻¹ epidermal growth factor (Sigma), 10 ng ml⁻¹ basic fibroblast growth factor (Peprotech), 4 μg ml⁻¹ Heparin, Pen Strep Glutamine (Gibco) and Gentamicin (Sigma); the cultures were maintained at 37° C./5% CO₂ for 15 days; then fixed using ice-cold acetone/methanol (1:1), visualized using Giemsa staining (Merck) and the number of mammary CFCs was enumerated.

### MPA/DMBA Tumourigenesis Protocol

7 weeks after receiving tamoxifen, a single MPA slow release pellet (Innovative Research of America) was implanted subcutaneously into mice. The mice were allowed to recover for 10 days and then, 1 mg of DMBA (Sigma) was administered orally; this was followed by three further doses of 1 mg of DMBA weekly. Mice were then examined weekly for tumour incidence and collected 11 weeks after the last DMBA administration. Control Bcl11a^{CreERT2}; tdTom animals which did not receive tamoxifen but were subjected to the same tumourigenic protocol, as well as littermates Bcl11a^{WT}; tdTom exposed to both tamoxifen and the MPA/DMBA protocol, had no detectable tdTomato^{pos} cells, confirming that the labelling and expansion of tdTomato cells is not an artefact of the MPA/DMBA protocol.

### Optical Tissue Clearing and Wholemount Immunostaining

Mammary tissue was dissected and cut into large pieces (˜15×15×2 mm) for immunostaining and clearing. CUBIC-based tissue clearing²⁷ was performed as previously described (Breast Cancer Res. 18, 127 (2016) and Nat. Commun., 7, 13053 (2016)) and combined with wholemount immunolabelling for visualization of tdTomato cells. Briefly, CUBIC Reagent 1A was prepared using urea (Sigma, 10% (w/w)), N,N,N′,N′-tetrakis(2-hydroxypropyl)ethylenediamine (Sigma, 5% (w/w)), triton-X100 (VWR, 10% (w/w)) and NaCl (Sigma, 25 mM) in distilled water. CUBIC Reagent 2 was prepared using sucrose (Fisher Scientific, 44% w/w), urea (Sigma, 22% w/w), 2,2′, 2″-nitrilotriethanol (Sigma, 9% w/w) and Triton X-100 (VWR, 0.1% w/w) in distilled water. Tissues were immersed in CUBIC Reagent 1A at 37° C. for 2-3 days. For immunostaining, samples were washed and subsequently blocked in PBS containing triton-X100 (0.5% (w/v)) and goat serum (10% (v/v)) overnight at 4° C. Primary antibodies were diluted in blocking buffer at 4° C. for 4 days with gentle rocking. Tissue was washed (3×1 h) and incubated with Alexa Fluor conjugated secondary antibodies for 2 days, washed in PBS and transferred to CUBIC Reagent 2 at 37° C. for at least 1 day for refractive index matching. Samples were immersed in CUBIC Reagent 2 for imaging and were imaged within 2 weeks. The following primary antibodies were used for immunostaining: rat anti-EpCAM-AF647 (Biolegend, clone G8.8, 1 μg ml⁻¹, 1:250); rabbit anti-K5 (Covance, PRB160P, 1:100). The following Alexa Fluor-conjugated secondary antibodies were purchased from Thermo Fisher Scientific and used 1:500: goat anti-mouse 488 (A11001); goat anti-rat 647 (A21247).

Immunofluorescence 5 μm sections of mammary glands were either stained with haematoxylin and eosin (H&E) or immunostained with antibodies for Csn2 (sc-166530, Santacruz, 1:50) and RFP (600-401-379, Rockland, 1:200). Secondary staining involved, goat anti-rabbit 647, goat anti-rat 647, or anti-mouse 488, anti-rabbit 594 (1:200, Invitrogen). Nuclear stain was detected using ProLong Gold Antifade Mountant with DAPI (Thermofisher, P36941).

### Confocal Microscopy and Image Analysis

Images of wholemount mammary glands were acquired using a Leica TCS SP8 and SP5 inverted confocal microscopes with 10×/0.4 or 40×/1.3 HC PL APO objective lenses. H&E images were acquired using a Zeiss Axioplan 2 microscope. Laser power, line averaging and step increment were adjusted manually to give optimal fluorescence intensity for each fluorophore with minimal photobleaching. Image overview and 3D reconstructions were generated using ImageJ software.

### Differential Expression

Differential gene expression analysis was performed using edgeR (Bioinformatics, 26, 139-140 (2010)). For pairwise comparisons between clusters, genes with a mean expression level below 0.1 were removed from the analysis. A negative binomial generalised log-linear model was fitted to the remaining genes with the cluster assignments as covariate(s). The ‘treat’ function with “trend=TRUE, robust=TRUE” was used to identify genes that have a significantly higher log fold change than log 2(1.5) at an FDR of 0.01. The marker genes used for cell type inferences were identified using the ‘findMarkers’ function in scran with default settings.

### Western Blot

Cells were lysed using RIPA (Cell signalling) and protease inhibitors (Roche) as per manufacturer instructions. For tissue lysis, mammary tissue was pulverised using liquid nitrogen, a mortar and a pestle. The pulverised sample was dissolved in RIPA after which the sample was passed through a 25G needle. Total protein was measured using the bicinchoninic acid (BCA) method (Pierce Biotechnology). In total, 50 μg cell lysate was separated using 7.5% SDS-PAGE gels and transferred to PVDF membranes by electro-blotting. Membranes were blocked in 5% (w/v) milk in Tris-buffered saline containing 0.1% Tween-20 (TBST). Blots were then incubated at 4° C. overnight with primary antibodies as indicated, washed in TBST and subsequently probed with secondary antibodies for 1 h at room temperature. ECL solution was then added to the membrane and analysed. Antibodies used were, BCL11A (A300-380A, Bethyl, 1:1000), WAP (se-398276, Santa Cruz, 1:1000) and β-actin (sc-130656, Santa Cruz, 1:1000).

### Liquid Chromatography-Mass Spectrometry (LC-MS)

An equal volume of 6M GuHCl was added to the serum and mixed. Organic solvent (75% ACN) was then added and the samples mixed. Samples were centrifuged and the supernatant transferred and evaporated under OFN. The extract was reconstituted into 0.1% formic acid and loaded onto a Waters HLB Micro elution plate and extracted by SPE. The eluant was evaporated and reduced and alkylated then digested and analysed by LC-MS using a nano LC system. Peptides were analysed using a Q-Exactive Plus orbitrap using a PRM and IDA based analysis.

### Statistical Significance

All P values were calculated using Student's t-test unless otherwise indicated in the figure legends.

### Results

The role of Bcl11A in alveolar development

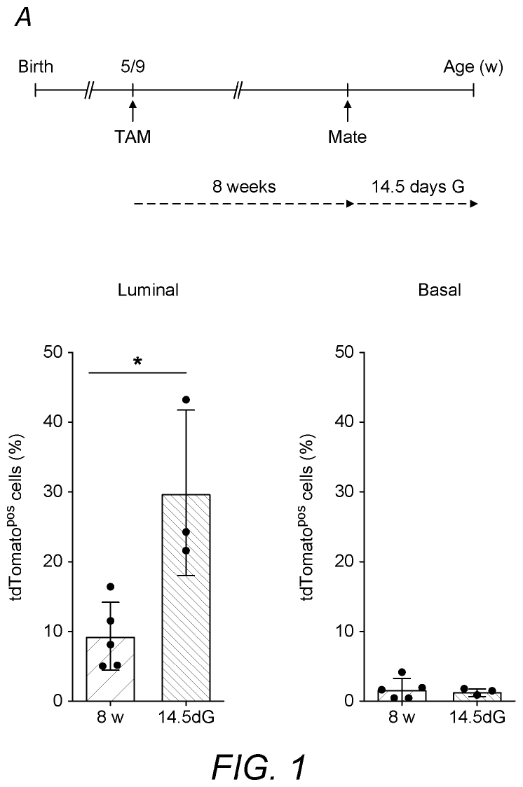

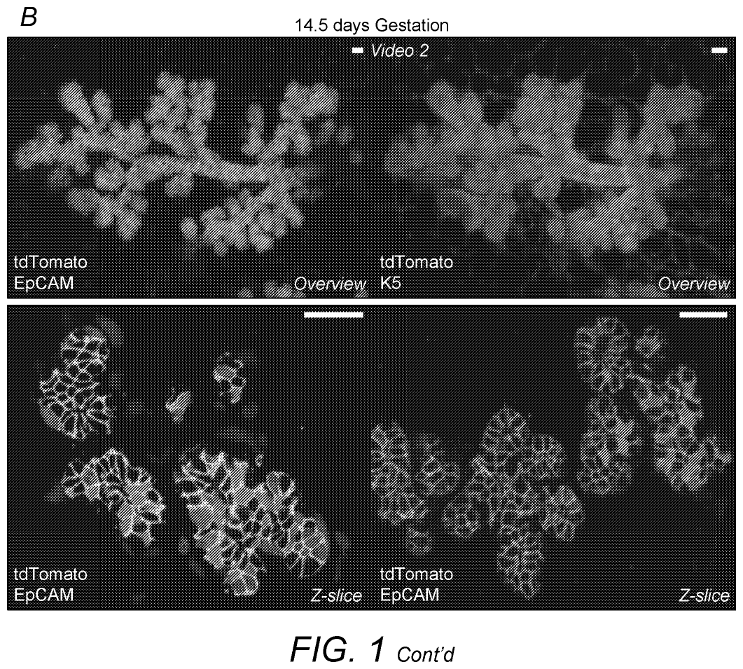

To assess if Bcl11a expressing cells are partial to alveolar differentiation and TNBC tumour initiation, a Bcl11a reporter mouse was generated by knocking-in an IRES-CreERT2 cassette into the 3′ UTR region of Bcl11a, which is referred to herein as Bcl11a^{CreERT2}. These mice allowed lineage tracing to be performed when crossed to the Rosa26-LSL-tdTomato reporter mice (tdTom). Tamoxifen mediated CreERT2 activation induced tdTomato expression in Bcl11a expressing cells and any of their future progeny (FIG. 1A). Fluorescence-activated cell sorting (FACS) analysis one day post-labelling revealed that the vast majority (95%±1.6) of tdTomato^{pos} cells reside within the luminal compartment and are primarily luminal progenitors (FIG. 1A).

To investigate if Bcl11a-labelled cells contributed to the alveologenesis process we mated females 8-week post tamoxifen injection and analysed the mice at 14.5 days gestation. We found that the number of tdTomato^{pos} luminal cells expanded significantly to constitute approximately 30% of the luminal compartment and analysis after natural weaning reveals persistence of labelled cells, at levels similar to nulliparous mice (FIG. 1B).

The above data supports a role of Bcl11a in alveolar production.

### Bcll11a Expression is Detection in Early Stage Tumour Genesis of TNBC Model

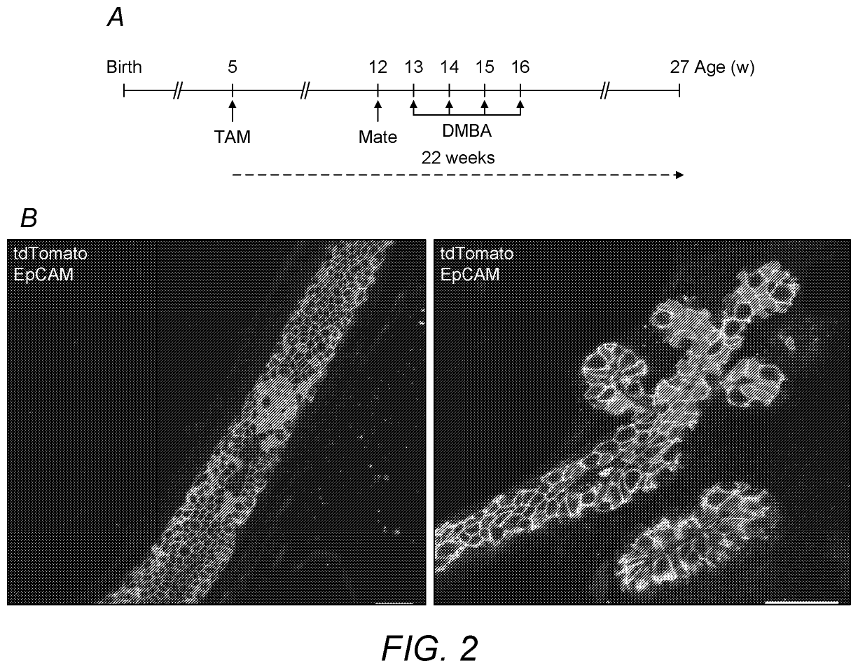

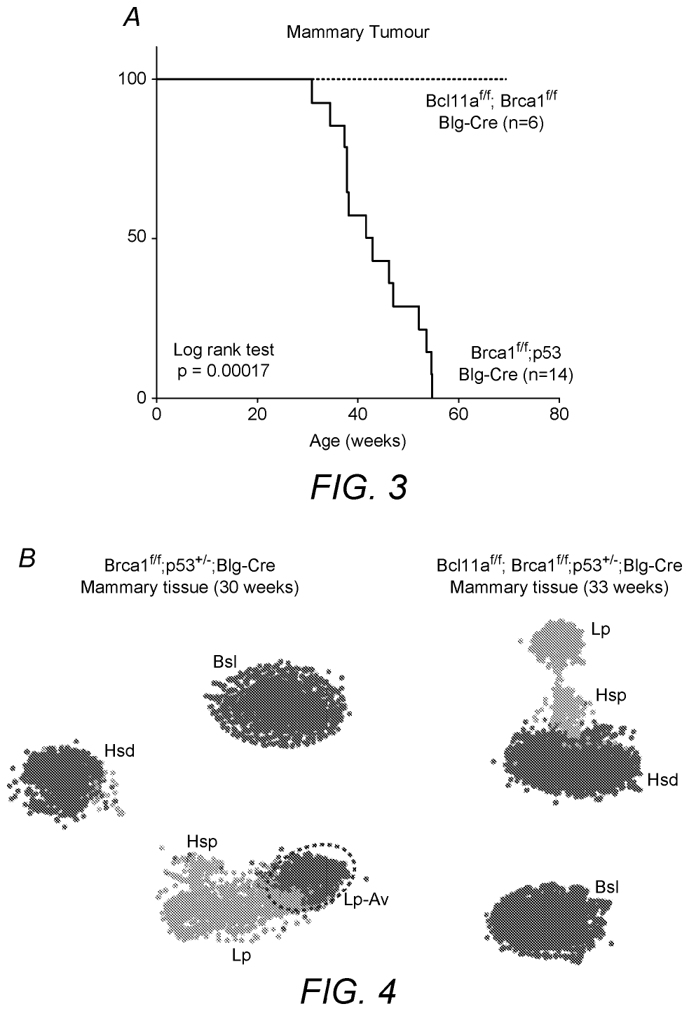

To examine the role of long lived tdTomato^{pos} cells in the early stages of TNBC development, the Bcl11a^{CreERT2}; tdTom mice were subjected to the MPA/DM BA protocol of TNBC tumourigenesis (FIG. 2A) (Genome Biol., 8, R76 (2007) and Nature, 468, 103-107 (2010)), which has been previously shown to be Bcl11a dependent (Nat. Med., 15, 907-913 (2009)). Tile-scans of the glands at a pre-cancerous stage, 11 weeks post DMBA administration, revealed an expansion of the tdTomato^{pos} cells and the appearance of alveolar-like structures, and, in some instances, the expansion led to the formation of abnormal structures which grew and invaded the lumen (FIG. 2B).

### Bcll11a K/O Mouse do not Develop TNBC

To investigate how driver oncogenes modulate the fate of luminal progenitor cells in TNBC, the Brca1/p53 TNBC mouse model (Brca1^{f/f}; p53^{+/-}; Blg-Cre) that harbours a conditional Brca1 loss-of-function in the luminal progenitor compartment, was used, with focus on the TNBC oncogene BCL11A¹. Deletion of Bcl11a in luminal progenitors was found to fully protect Brca1/p53 mice from developing mammary tumours (FIG. 1A) suggesting that Bcl11a is essential for Brca1 mediated TNBC.

### RNASeq of Mammary Tissues Identify Alveolar Like Cells in Cancer Bearing Mice (Bcl11A K/O) but not in Diseases Free Animals (Bcl11A WT).

RNASeq of mammary tissues at week 30 in tumour (Bcl11a positive) and at week 33 non-tumour (Bcl11a deletion) bearing animals (33 weeks is before any physical evidence of tumour). Clustering analysis reveal 4 common cellular populations between tumour vs. non tumour animals. Lp; Luminal Progenitors, Hsp; hormone sensing progenitors, Hsd; hormone sensing differentiated & Bsl; Basa. In the tumour bearing animals there was a population of Lp-Av (alveolar cells).

This suggests in early tumour development (before any physical sign) there is a population of alveolar cells not present in disease free animals.

Mammary Gland Specific Over Expression of Bcl11A Drives Alveologenesis; these Structures can Also be Observed in Early Stage Tumour Bearing Non-Pregnant Animals.

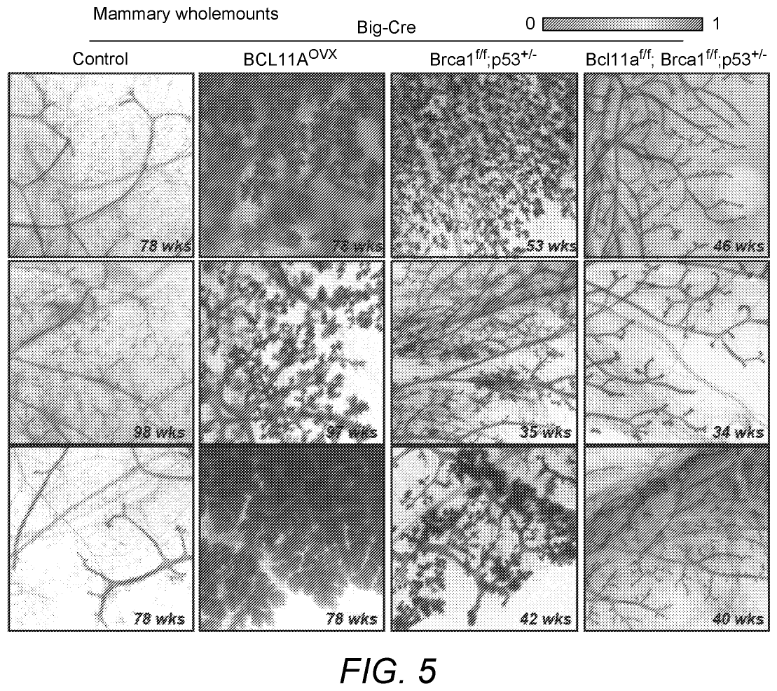

To identify the cellular events underpinning this protective effect of Bcl11a deletion on the Brca1/p53 model, scRNAseq on mammary epithelial cells from nulliparous Brca1/p53 and Bcl11a^{f/f}; Brca1/p53 mice at 30-33 weeks of age was performed, before tumours were detected. It was found that the Brca1/p53 mammary epithelium phenotypically resembles that of a mouse during gestation (FIG. 5) which is in agreement with a previous report of precocious mammary alveologenesis in a different model of Brca1/p53 (Science (80-.). 314, 1467 LP-1470 (2006)).

scRNAseq and wholemount analysis of the Bcl11a^{f/f}; Brca1/p53 mice revealed that the aberrant differentiation behaviour of the luminal progenitors was completely abolished upon the deletion of Bcl11a (FIG. 5).

The above results suggest that, in the early stages of TNBC, Bcl11a-dendent alveolar formation occurs.

### Detection of Aberrant Differentiation on MPA/DMBA Treated Animals

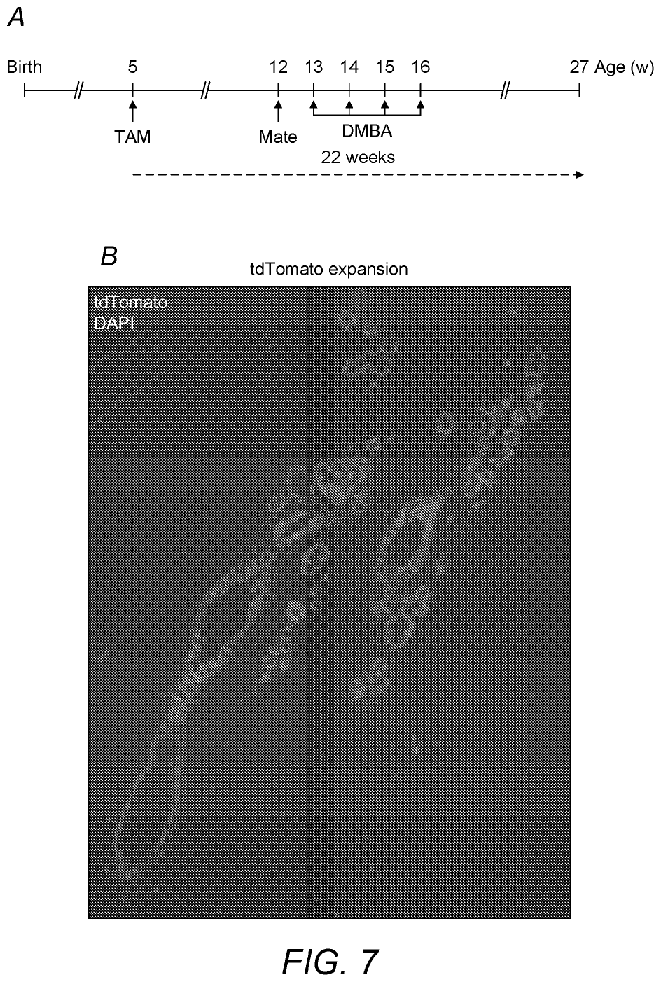

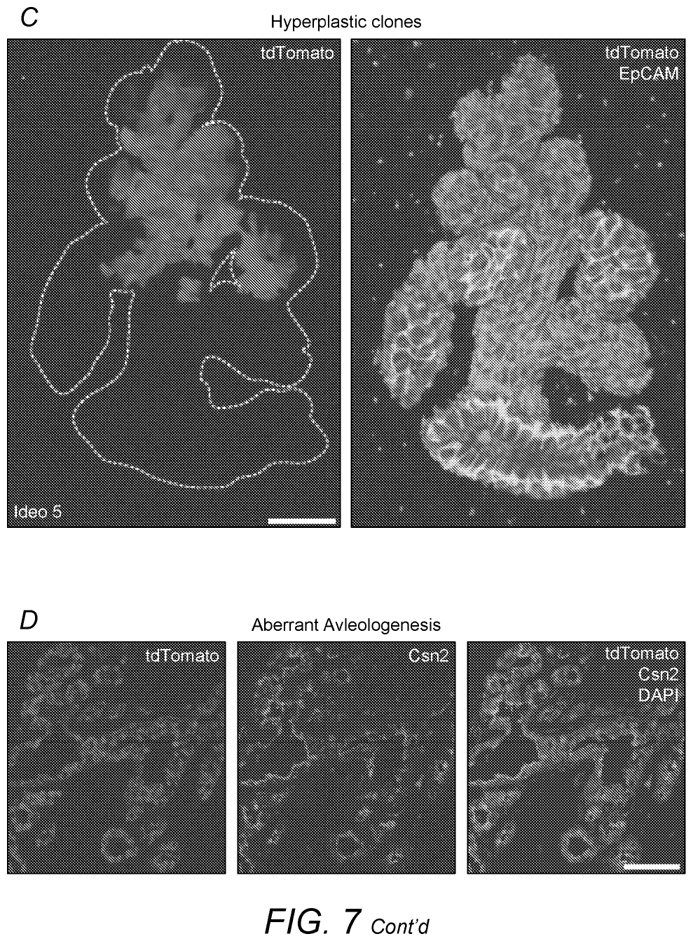

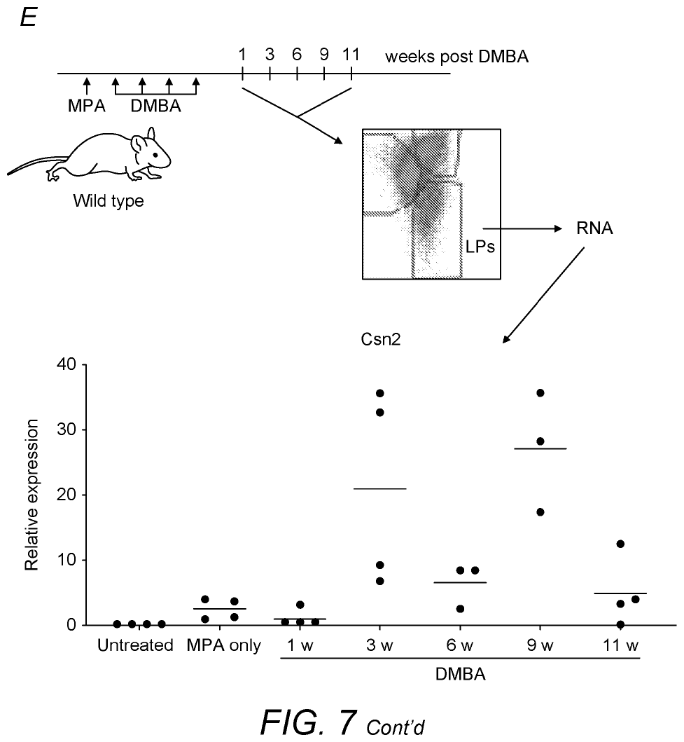

The role of long lived tdTomato^{pos} cells in the early stages of TNBC development was next examined. Bcl11a^{CreERT2}; tdTom mice were subjected to the MPA/DMBA protocol of TNBC tumourigenesis (FIG. 7A) (Genome Biol., 8, R76 (2007) and Nature, 468, 103-107 (2010)), which has previously been shown to be Bcl11a dependent (Nat Commun., 6, 5987 (2015)). Tile-scans of the glands at a pre-cancerous stage, 11 weeks post DMBA administration, revealed an expansion of the tdTomato^{pos} cells and the appearance of alveolar-like structures (FIG. 7B). In some instances, the expansion led to the formation of abnormal structures which grew and invaded the lumen (FIG. 7C). 3D imaging of these structures revealed a resemblance to atypical ductal hyperplasia and ductal carcinoma in situ (FIG. 7C). IHC analysis of these mice revealed the appearance of milk producing cells, which is compatible with the appearance of alveolar-like structures and in agreement with the observations made in the Brca1/p53 model (FIG. 7D). RNA was extracted from luminal progenitor cells that were FACS isolated at various timepoints following MPA/DMBA treatment. FIG. 7E shows Csn2 expression in the various samples. MPA only mice received MPA three weeks prior to sample collection. Quantification was performed by first normalising against Gapdh levels and relative to MPA only samples.

The above results show that Csn2 expression was observed in luminal progenitor cells 3 weeks post treatment, whereas tumours were not observed until circa. 12 to 14 weeks post treatment. These results thus demonstrate that the present invention, and in particular the detection of aberrantly differentiated luminal progenitor cells and/or Csn2 in the breast of a subject, is an excellent method for detecting the early signs of breast cancer (e.g. TN BC).

Detection of CSN2 and/or WAP

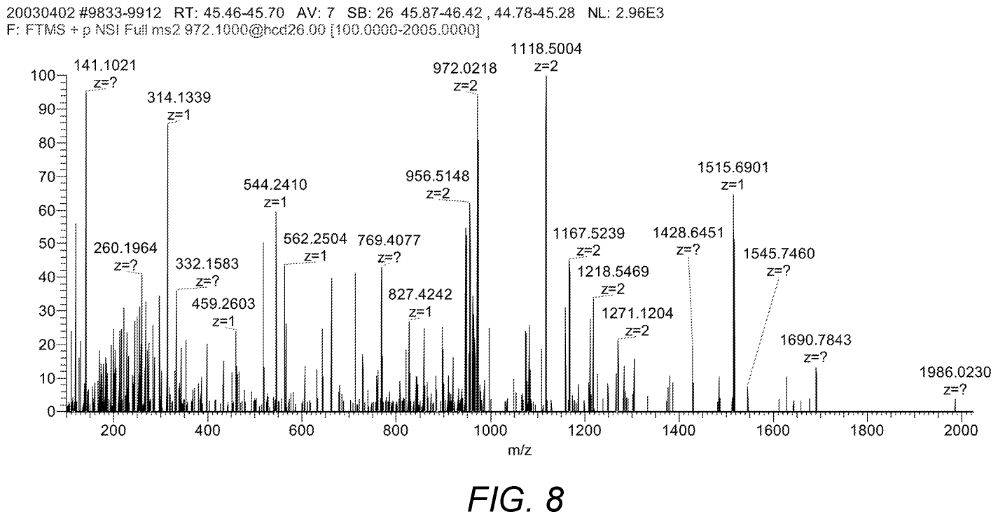

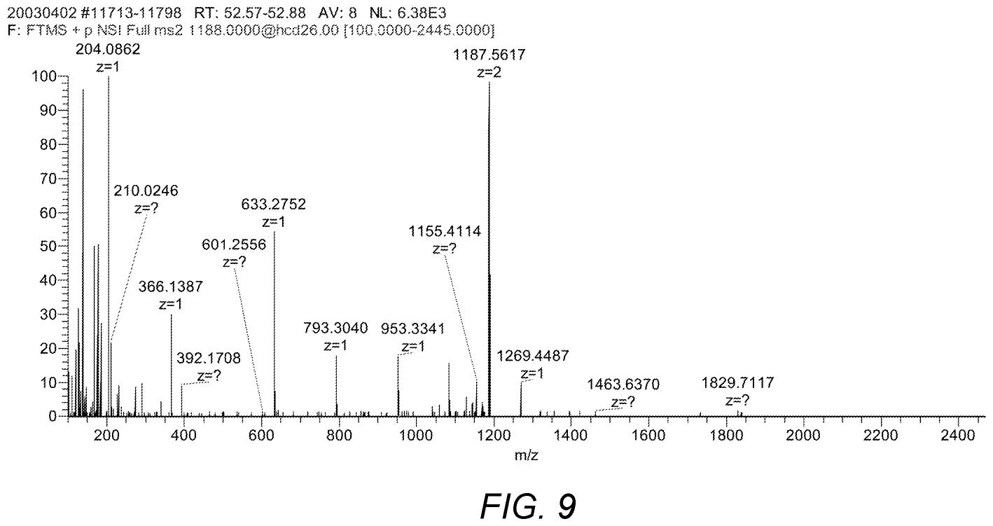

Surprisingly, it has been found that LC-MS analysis of mammary gland tissue from Brca1/p53 mice can detect peptides matching CSN2 and WAP (FIGS. 8 and 9). This correlates with the mice displaying the aberrant differentiation of luminal progenitors described above.

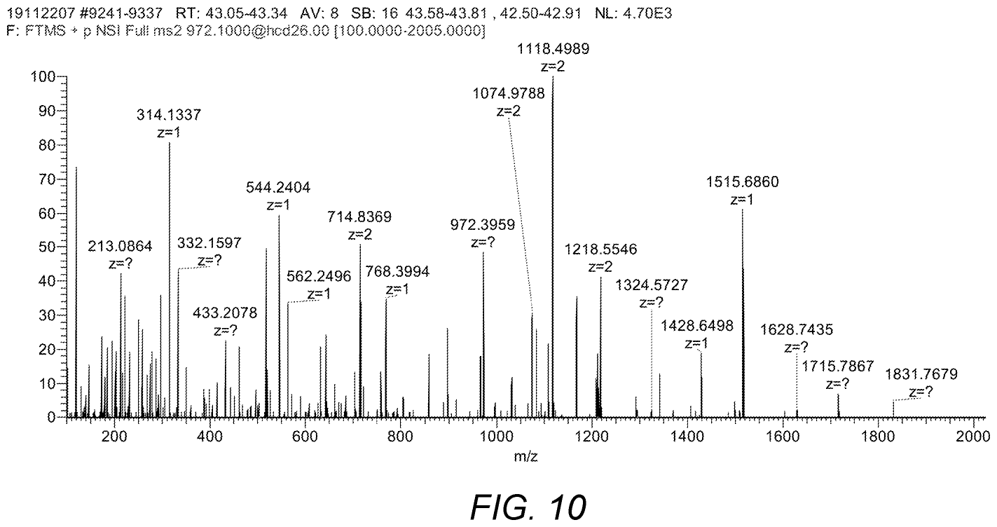

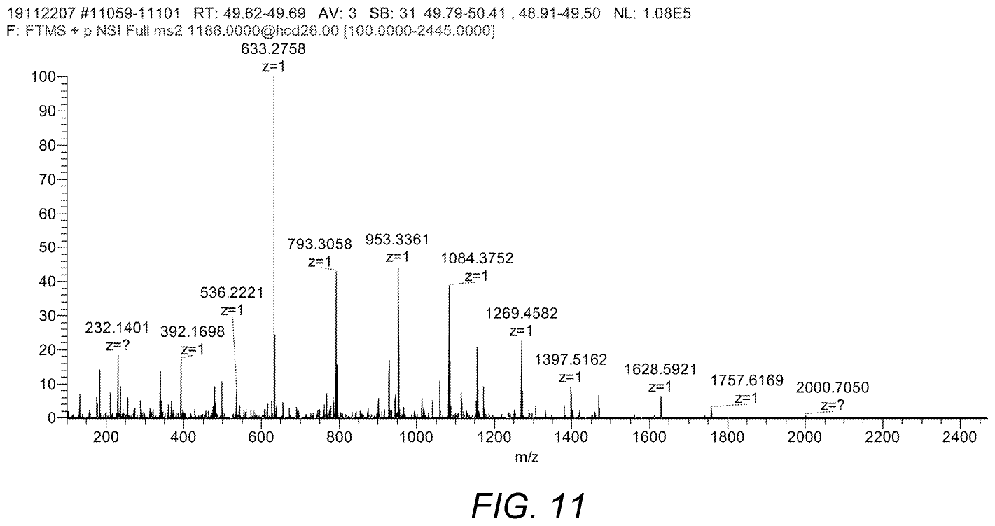

Furthermore, the same peptides can be detected, using LC-MS, in the blood of lactating WT mice, indicating that these proteins can enter the blood (FIGS. 10 and 11). These milk protein peptides are undetectable (using LC-MS) in blood serum from male mice. Hence, these peptides can be used for the early detection of aberrant luminal differentiation in women with Brca1 pathological variants (PV) using more sensitive methods.

RNA which codes for CSN2 has also been detected, using qPCR, in the blood of Brca1/p53 mice. The same RNA was undetectable in the blood of male mice (using qPCR).

### Human Data

RNA was extracted from mammary luminal progenitors that were FACS isolated from either: i) control patients; or ii) BRCA1 carrier patients. CSN2 expression in these human samples was then determined. Quantification was performed by first normalising against GAPDH levels and relative to control STG218.

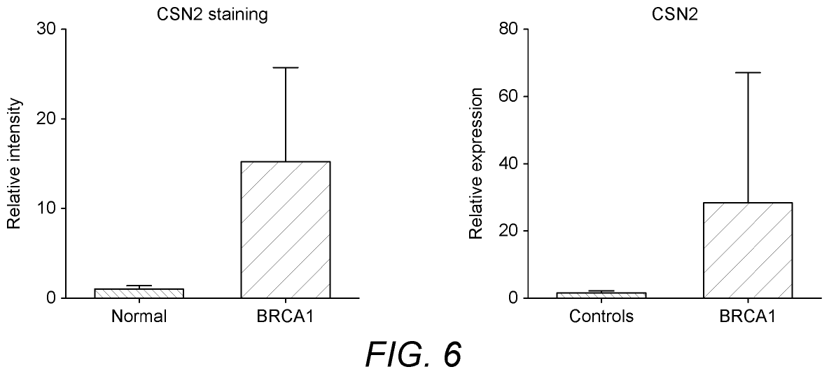

FIG. 6 shows the average protein expression of CSN2 in BRCA1 mutation carriers and non-BRCA1 carriers (normal) measured by IHC (FIG. 6A), and the average RNA expression of CSN2 in BRCA1 mutation carriers and non-BRCA1 carriers (normal) measured by qPCR (FIG. 6B).

1. A method of assessing the likelihood of a subject having breast cancer or a precancerous condition associated with breast cancer, said method comprising analysing the subject's breast to determine whether aberrantly differentiated luminal progenitor cells are present in the breast, wherein the presence of aberrantly differentiated cells in the breast is indicative of the subject having a high likelihood of having breast cancer or a precancerous condition associated with breast cancer.

2. A method according to claim 1, wherein the aberrantly differentiated cells are cells that aberrantly differentiate to form alveolar structures.

3. A method according to claim 1 or 2, wherein the method comprises analysing the subject's breast to determine whether alveolar structures are present in the subject's breast.

4. A method according to any one of claims 1 to 3, wherein the subject is a subject who has been identified as being at risk of developing triple negative breast cancer.

5. A method according to any one of claims 1 to 4, wherein the subject has a mutation of the BRCA1 and/or BRCA2 gene, for example a mutation of the BRCA1 gene, or has a family history of breast cancer, or is homologous recombination deficient.

6. A method according to any one of claims 1 to 5, wherein the analysis of the subject's breast comprises one or more techniques selected from:
a) scanning and/or imaging the breast to determine whether aberrantly differentiated cells are present; and/or
b) obtaining a sample of material from the breast of the subject, and analysing the material to determine whether a species associated with aberrantly differentiated cells is present.

7. A method according to claim 6, wherein the species associated with aberrantly differentiated cells is selected from the group consisting of proteins, DNA molecules and RNA molecules.

8. A method according to claim 7, wherein the species is selected the group consisting of a milk protein and a DNA or RNA molecule that codes for a milk protein.

9. A method according to claim 8, wherein the milk protein is selected from casein (e.g. beta-casein), alpha-lactalbumin, lactoferrin, immunoglobulin immunoglobulin A, lysozyme, whey acidic protein and serum albumin.

10. A method according to claim 9, wherein the milk protein is selected from beta-casein and whey acidic protein.

11. A method according to claim 10, wherein the milk protein is beta-casein.

12. A method according to claim 10, wherein the milk protein is whey acidic protein.

13. A method according to any one of claims 8 to 12, wherein the method further comprises analysing material obtained from the breast of the subject to determine whether a further species associated with aberrantly differentiated cells is present.

14. A method according to claim 13, wherein the further species associated with the aberrantly differentiated cells is DNA or RNA from a gene selected from BCL11A, ELF5, SOX10, FOXC1 Ki-67, HOXD13 and PCDHGB7, and any protein expressed from those genes.

15. A method according to any one of claims 7 to 14, wherein a protein is determined to be present in the sample using one or more techniques selected from enzyme-linked immunosorbent assay (ELISA), Western Blot analysis and mass spectrometry; and/or a DNA or RNA is determined to be present in the sample using one or more techniques selected from quantitative polymerase chain reaction (qPCR), RNA sequencing (RNAseq) and DNA sequencing (DNAseq).

16. A method according to any one of claims 6 to 15, wherein the sample of material from the breast of the subject is obtained from via the nipple of the subject's breast, or from surgery or biopsy.

17. A method according to any one of claims 6 to 16, wherein the sample is selected from a fluid which has been expressed from the breast of the subject.

18. A method according to any one of claims 6 to 17, wherein the sample of material is obtained from breast of the subject via a breast pump and/or a needle.

19. A method according to any one of claims 6 to 15, wherein the sample of material from the breast is obtained from a sample of blood taken from the subject.

20. A method according to claim 19, wherein the sample of blood is not taken from the breast of the subject.

21. A method according to claim 19 or 20, wherein the sample of material from the breast of the subject is obtained from a sample of blood taken from the subject, and the species associated with aberrantly differentiated cells is beta-casein (CSN2).

22. A method according to claim 6, wherein the breast of the subject is scanned and/or imaged using one or more techniques selected from magnetic resonance imaging (MRI), mammography, computerised tomography (CT) scanning, and/or contrast imaging.

23. A method according to any one of claims 1 to 22, wherein the subject is one who has undertaken a pregnancy test.

24. A method of treating breast cancer, said method comprising the steps of:
a) assessing the likelihood of the subject having breast cancer or a precancerous condition associated with breast cancer by a method as claimed in any one of claims 1 to 23; and
b) administering a standard breast cancer treatment regimen to the subject if the likelihood of the subject having breast cancer or a precancerous condition associated with breast cancer is assessed as being high.

25. A method according to claim 24, wherein the standard breast cancer treatment regimen is selected from surgery, chemotherapy and/or radiotherapy.

26. A method according to claim 24, wherein the standard breast cancer treatment regimen is selected from elective mastectomy and/or chemoprevention.

27. A method for monitoring the onset of breast cancer or a precancerous condition associated with breast cancer, or for monitoring the recurrence and/or growth of breast cancer in a subject, said method involving assessing the likelihood of the subject having breast cancer or a precancerous condition associated with breast cancer by a method as claimed in any one of claims 1 to 23 at two or more time points separated by a predetermined time interval.

28. A chemotherapeutic agent for use in the treatment of a subject in need of said treatment, the treatment comprising the steps of:
a) Assessing the likelihood of the subject having breast cancer or a precancerous condition associated with breast cancer by a method as claimed in any one of claims 1 to 23;
b) administering a chemotherapeutic agent to the subject if the likelihood of having breast cancer or a precancerous condition associated with breast cancer is assessed as being high.

29. A chemotherapeutic agent according to claim 28, wherein the chemotherapeutic agent is an anthracycline (e.g. doxorubicin or epirubicin) or a taxane (e.g. docotaxel or paclitaxel).
